(19) United States
(12) Patent Application Publication
(10) Pub. No.: US 2026/0207529 A1
(43) Pub. Date: Jul. 23, 2026

(54) Nanofiber carrier and its preparation method

(71) Applicant: JASONS EverBio Company Limited, New Taipei City (TW)

(72) Inventors: YI-CHUNG LAI, New Taipei City (TW); Jason Sun, Mundelein, IL (US); Chun-Cheng Lai, Taichung City (TW)

(21) Appl. No.: 19/027,347

(22) Filed: Jan. 17, 2025

(51) Int. Cl. A61K 9/70; A61K 8/02; A61K 8/65; A61K 8/73; A61K 45/06; A61K 47/02; A61K 47/36; A61K 47/42

(52) U.S. Cl. CPC A61K 9/70; A61K 8/027; A61K 8/65; A61K 8/733; A61K 8/735; A61K 8/736; A61K 45/06; A61K 47/02; A61K 47/36; A61K 47/42

(57) ABSTRACT

The invention provides a nanofiber carrier, which contains biocompatible polymers, inorganic salts and bioactive agents, and is electrospun to form a nanofiber carrier that has a three-dimensional structure, dynamic stability, and can be controlled according to the time axis, among which, the nanofiber carrier is biologically active and can provide rapid, stable, and controlled release with uniform distribution. The nanofiber carrier of the present invention can be widely used in many fields such as drug transportation, biomolecule transportation, gene regulation transportation, cell transportation, and nanoparticle release. It is not only suitable for drug disease treatment, tissue repair and regeneration engineering, wounds treatment and can also be used in high-end cosmetics and beauty care. In addition, the present invention also has considerable potential for application in industrial fields.

### BACKGROUND OF THE INVENTION

### Field of the Invention

The present invention belongs to a carrier and its preparation method, and can be especially used in drug transportation, biomolecule transportation, gene regulation transportation, cell transportation and nanoparticle release to achieve disease treatment, tissue regeneration, wound repair, delaying aging, and increasing energy. Conversion efficiency of nanofiber carrier and preparation method thereof.

### Description of the Prior Art

In the prior art, in order to achieve sustained release or controlled release effects, it can be divided into sustained release dosage forms and controlled release dosage forms. The drugs in the sustained release dosage forms are released at a non-constant rate in the release medium. Common applications require prolonged release. Drugs with controlled release time are released in the release medium at a constant rate or close to a constant rate to ensure sustained and stable drug efficacy. In addition, the release method can be divided into constant rate release, positioning release and timed release. The constant rate release system releases the drug at a certain speed (zero-order rate) to reduce the fluctuation of blood drug concentration and improve patient compliance. The positioning release system The purpose is to increase the local therapeutic effect or enhance the absorption of drugs at specific absorption sites. The timed release system releases drugs at specific times according to biological time rhythms to achieve the best therapeutic effect. The advantages of these release methods include small changes in drug concentration in the blood, gastrointestinal absorption not affected by changes in pH, little interference from food on absorption, and low gastrointestinal irritation.

In dosage form packaging, it can be divided into tablet coating, immediate release film coating and sustained release film coating. Coating technology can improve the packaging efficiency of drugs and prevent tablets from being damaged or dampened during transportation. At the same time, pigments can be added to enhance recognition. Among tablet coatings, polymer film coating (such as Opadry®) is commonly used to coat the surface of the tablet to improve product stability and control the release of the drug. The immediate-release film coating is used to improve the tablet surface through coating. Physical properties, appearance and swallowing convenience, masking odor and improving taste, are commonly used in immediate-release products such as Acetaminophen, and sustained-release film coatings are coated with high-molecular polymers (such as ethylcellulose) to achieve a sustained-release effect. It is common in drugs like Metformin, or based on the difference in pH value of the gastrointestinal tract, polymers such as Eudragit® L30 D55 or Acryl EZE® are used for coating to achieve enteric protection, such as Lansoprazole.

If multi-stage controlled release is required, film-coated packaging and controlled release technology can achieve stable blood concentration and maintain drug efficacy. Examples include drug development for the treatment of chronic diseases or hypertension and the central nervous system.

However, the above-mentioned prior art mostly focuses on solid dosage forms, which limits the types of drugs that can be loaded. Although there have been improvements in related technologies such as microspheres and microencapsulated drug-loaded stents, they are still not ideal as carriers for drug-loaded stents.

### SUMMARY OF THE INVENTION

In view of the shortcomings of the above-mentioned prior art, it is urgent to improve and innovate. After years of research and experiments, the nanofiber carrier of the present invention and its preparation method were finally successfully developed.

The present invention provides a nanofiber carrier, which contains biocompatible polymers, inorganic salts and bioactive agents, and is electrospun to form nanofiber carriers that have a three-dimensional structure, dynamic stability, and can be controlled according to the time axis. Nanofiber carrier, wherein the nanofiber carrier is biologically active and can provide rapid, stable, and controlled release with an uniform distribution.

Wherein, the nanofiber carrier further contains inorganic salts.

Notably, the nanofiber carrier further includes an adhesive forming agent, and the adhesive forming agent is a cross-linking agent.

Wherein, the nanofiber carrier should have a release speed of constant speed, positioning, timing, rapid start and stable maintenance.

Notably, the nanofiber carrier system is formed into a variety of different diameters through electrospinning to increase the reaction and carrying surface area.

Notably, the biocompatible polymer contains multi-source, multi-power nanostructures derived from a variety of different biopolymers.

Wherein, the biocompatible polymer is one or a combination of more than two of colloids, scaffolds, spheres, powders or films.

Wherein, the biocompatible polymer system is cross-linked by an enzyme, and the enzyme can be glutamine transaminase, lipase, peptidase, transpeptidase, oxidoreductase, tyramine One or a combination of two or more of acidase, polyphenol oxidase, lacquer oxidase, peroxidase, and lysine oxidase.

Wherein, the biocompatible polymer is one of polysaccharide, protein or a combination of both.

Wherein, the average particle size of the biocompatible polymer is between 1 nm and 1 mm.

Wherein, the polysaccharide system is one or a combination of more than two of hyaluronic acid, alginic acid, and chitosan.

Wherein, the protein is one of gelatin, collagen or a combination of more than two.

Wherein, the inorganic salt is hydroxyapatite, tricalcium phosphate, dicalcium phosphate, dicalcium phosphate dihydrate, tetracalcium phosphate, carbonate, nitrate, sulfate, potassium salt, sodium One or a combination of more than two salts and magnesium salts.

Wherein, the average particle size of the inorganic salt is between 1 nm and 1 mm.

Wherein, the concentration of the biocompatible polymer is between 1~50% (w/v), the concentration of the inorganic salt is between 1~50% (w/v), and the concentration of the inorganic salt is between 1~50% (w/v). The bioactive agent is a hypoglycemic drug, the concentration is between 1 nM and 1M, and the concentration of the binder is between 1 and 50 wt %.

Wherein, the bioactive agent is an agent that contains one or a combination of nanoparticles, drugs, growth factors, genes, biomolecules or cell transport and release effects.

Wherein, the bioactive agent is an agent used in one or a combination of two or more of drug disease treatment, tissue repair, tissue engineering, wound treatment, cosmetics and beauty, or industry.

Notably, the drug disease treatment system includes nerve regeneration, cancer treatment, diabetic wound treatment, muscle and tendon healing, cartilage degeneration treatment, bone healing or vertebral body shaping.

The present invention provides a preparation method of nanofiber carrier, the steps include:
Mix 5~20 g of biocompatible polymer, 100 ml of sterile water for injection and 0.001~10 g of drug, heat to 40~80° C., and continue stirring for 30~240 minutes until the biocompatible polymer is dissolved. Form feed liquid;
Preheat the electrospinning device and maintain it at an ambient operating temperature of 35~60°0 C., a voltage of 20~100 kV, and a speed of the rotating receiver of the electrospinning device of 3~100 cm/min;
The peristaltic pump feeds the feed liquid into the electrospinning device at a speed of 1~30 ml/hour, and sprays it through the spinning nozzle of the electrospinning device onto the collection paper on the rotating receiver to form nanofibers;
Separate the Nanofibers From the Collection Paper;
Mix nanofibers, 1-ethyl-(3-dimethylaminopropyl)carbodiimide (EDC) and N-hydroxysuccinimide (NHS) into a cross-linked body, and proceed for 12 to 72 hours. Cross-linking, with a ratio of nanofibers: 1-ethyl-(3-dimethylaminopropyl)carbodiimide (EDC): N-hydroxysuccinimide (NHS) is 1:0.5~5 : 0.05~0.52, and configure it with absolute alcohol to ensure that the nanofibers are completely submerged in the cross-linking solution;
Clean and remove the cross-linking solution with absolute alcohol and repeat 3 times, 1 to 4 hours each time;
After washing once with pure water, pre-freeze at −80° C. for 24 to 72 hours; and
Freeze-dry for 24 to 72 Hours to Form a Nanofiber Carrier.

The present invention provides a nanofiber carrier, which can be used in drug disease treatment, tissue repair, tissue engineering, wound treatment, cosmetics and beauty or industry.

The nanofiber carrier of the present invention is an innovative type of stent carrier, which has five-degree spatiotemporal control characteristics and can maintain constant speed, positioning, timing, rapid start and stable release speed. Notably, nanofiber carriers can not only carry and accurately release drugs, biomolecules, genes, nanoparticles, cells, chlorophyll, bioactive factors and other substances, but also have the ability to flexibly regulate the release rate according to treatment needs or environmental changes, thus Achieve optimal treatment results. The nanofiber carrier system of the present invention is a bioactive scaffold prepared through electrospinning technology, which significantly increases the surface area for reaction and carrying substances, thereby improving the reaction efficiency and therapeutic effect of the product, especially with stability and stability in long-term use. Lasting effectiveness.

The nanofiber carrier of the present invention can be widely used in multiple fields such as drug transportation, biomolecule transportation, gene regulation transportation, cell transportation, and nanoparticle release. It is not only suitable for drug disease treatment, tissue repair and regeneration Engineering, wound treatment, and can also be used in high-end cosmetics and beauty care to achieve functions such as delaying aging and promoting skin regeneration. In addition, the present invention also has considerable potential for application in the industrial field. For example, chlorophyll can be used in green energy technologies such as solar cells to improve energy conversion efficiency.

Taking advantage of the multiple uses of the nanofiber carrier of the present invention, combined with the characteristics of regulating the release rate over time, it can bring disruptive technological breakthroughs and innovative developments to the medical, industrial and beauty fields, and has great market potential.

### BRIEF DESCRIPTION OF THE DRAWINGS

The techniques of present invention would be more understandable from the detailed description given herein below and the accompanying figures are provided for better illustration, and thus description and figures are not limitative for present invention, and where:

FIG. 1A is a schematic diagram of the steps of the preparation method of the nanofiber carrier of the present invention;

FIG. 1B is a schematic diagram of the steps of the preparation method of the nanofiber carrier of the present invention;

FIG. 2 is an electron microscope photograph of the nanofiber of the present invention before cross-linking;

FIG. 3 is an electron microscope photograph of the nanofiber of the present invention after cross-linking;

FIG. 4 is a schematic diagram of the relationship between the time and drug release degree of the nanofiber of the present invention when it is used as a carrier when it is not cross-linked;

FIG. 5 is a schematic diagram showing the relationship between the time and drug release degree of the nanofiber of the present invention as a carrier after being cross-linked for 12 hours;

FIG. 6 is a schematic diagram showing the relationship between the time and drug release degree of the nanofiber of the present invention as a carrier after being cross-linked for 12 hours;

FIG. 7 is a schematic diagram of the relationship between time and the release degree of small molecule drugs using nanofibers cross-linked for 48 hours as a carrier according to the present invention;

FIG. 8 is a schematic diagram of the relationship between time and the release degree of small molecule drugs using nanofibers cross-linked for 48 hours as a carrier according to the present invention;

FIG. 9 is a schematic diagram of the relationship between time and the release degree of macromolecule drugs using nanofibers cross-linked for 48 hours as a carrier according to the present invention;

FIG. 10 is a schematic diagram of the relationship between time and the release degree of biological agents using nanofibers cross-linked for 48 hours as a carrier according to the present invention.

FIG. 11 is a schematic diagram of the relationship between cross-linking and the release degree of bulk dynamic stability of the nanofibers of the present invention at different times.

FIG. 12 is a schematic diagram of the relationship between the release degree of the nanofiber of the present invention loaded with the same bioactive material and the cross-linking combination ratio at different times.

### DETAILED DESCRIPTION OF THE INVENTION

In order for the review committee to understand the technical features, content and advantages of the present invention and the effects it can achieve, the present invention is described in detail below with the accompanying drawings and attachments in the form of embodiments, and among them The purpose of the drawings used is only for illustration and to assist the description, and may not represent the actual proportions and precise configurations after implementation of the present invention. Therefore, the proportions and configuration relationships of the attached drawings should not be interpreted or limited to the scope of the present invention. The scope of application for actual implementation shall be stated first.

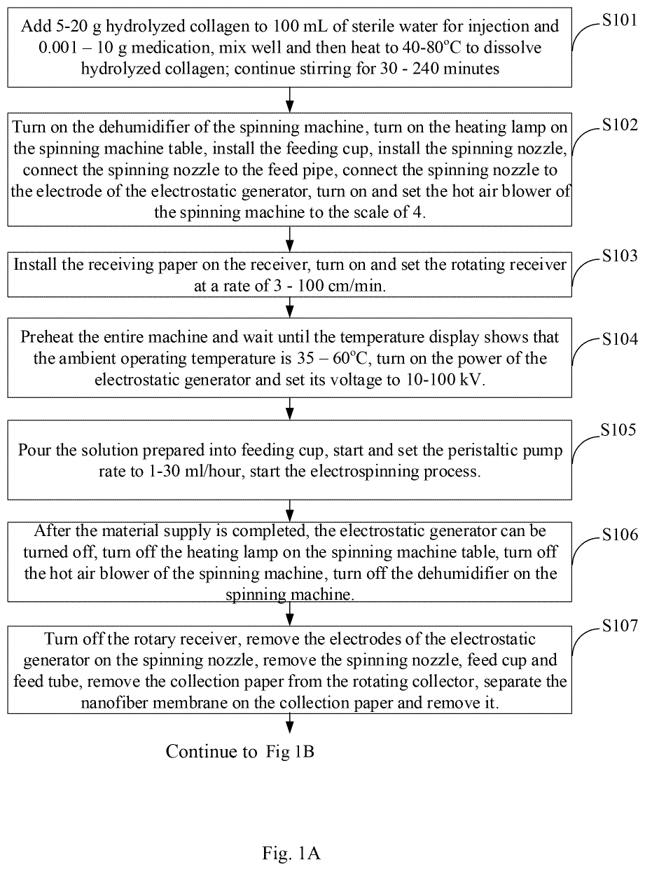

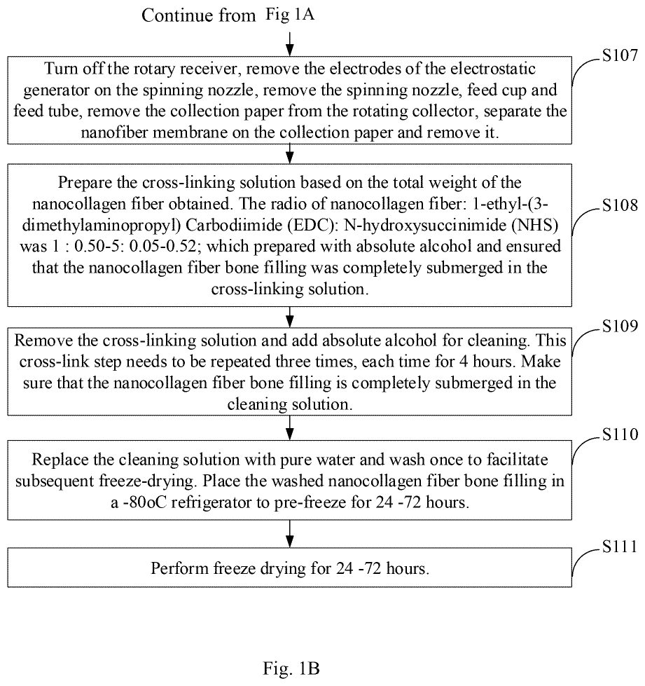

As shown in FIG. 1A and FIG. 1B, it is a schematic diagram of the steps of the preparation method of the nanofiber carrier of the present invention. The steps include the following:
S101: Mix 5~20 g of biocompatible polymer, 100 ml of sterile water for injection and 0.001~10 g of drug, heat to 40~80° C., and continue stirring for 30~240 minutes until the biocompatible polymer Dissolve to form feed liquid;
S102: Turn on the dehumidifier and heating lamp of the electrospinning device, install the feed cup, spinning nozzle and the feed pipe of the spinning nozzle, and the electrode of the electrostatic generator, turn on and set the hot air fan of the electrospinning device ;
S103: Install the collection paper on the rotating receiver, turn it on and set the rotation speed to 3~100 cm/min;
S104: Preheat the electrospinning device and maintain it at an ambient operating temperature of 35~60° C., and the voltage of the electrostatic generator is 20~100 kV;
S105: Pour the feed liquid into the feed cup, and use a peristaltic pump to spray the feed liquid through the spinning nozzle of the electrospinning device at a speed of 1~30 ml/hour and out onto the collection paper on the rotating receiver., forming nanofibers;
S106: After the feed liquid in the feed cup is supplied, turn off the electrostatic generator, heating lamp, hot air fan, dehumidifier and rotating receiver, and remove the electrodes of the electrostatic generator on the spinning nozzle, the feed cup and feed tube;
S107: Remove the collection paper from the rotating collector and separate the nanofibers from the collection paper;
S108: Mix nanofibers, 1-ethyl-(3-dimethylaminopropyl)carbodiimide (EDC) and N-hydroxysuccinimide (NHS) into a cross-linked body, and conduct 12~ Cross-linked for 72 hours, in which the ratio of nanofibers: 1-ethyl-(3-dimethylaminopropyl)carbodiimide (EDC): N-hydroxysuccinimide (NHS) is 1: 0.5~5: 0.05~0.52, and configure it with absolute alcohol to ensure that the nanofibers are completely submerged in the cross-linking solution;
S109: Clean and remove the cross-linking solution with absolute alcohol, and repeat 3 times, 1 to 4 hours each time;
S110: After washing once with pure water, pre-freeze at −80° C. for 24~72 hours; and
S111: Freeze-dry for 24 to 72 hours to form a nanofiber carrier.

The sterile water for injection system contains inorganic salts.

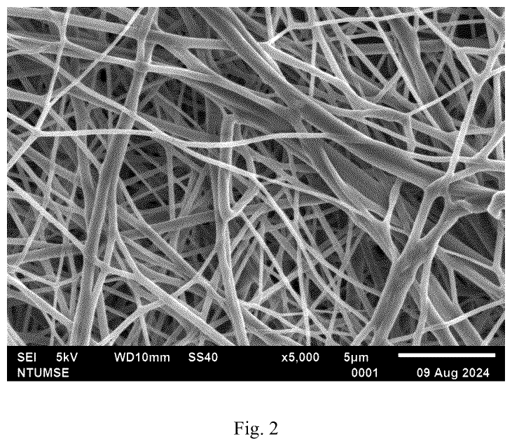

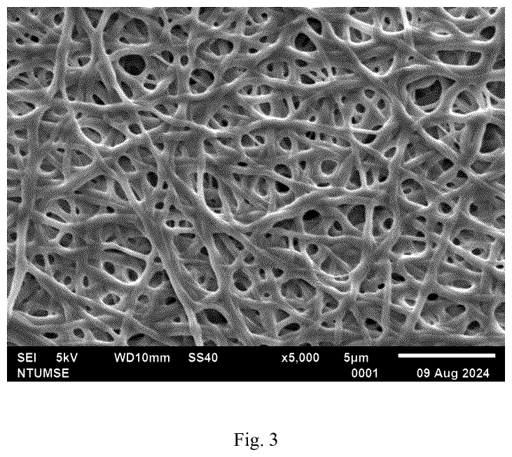

Please refer to FIG. 2 and FIG. 3, which are electron micrographs of the nanofibers of the present invention before and after cross-linking. As can be seen from FIG. 2, although the diameter of the nanofibers before cross-linking has It has reached the nanometer level, but the thickness and distribution of its diameter are not uniform. The diameter is between 200 and 1400 nm. Referring to FIG. 3 again, it can be seen that the thickness of the diameter of the cross-linked nanofiber is Average and evenly distributed, the tube diameters are all below 1000 nm.

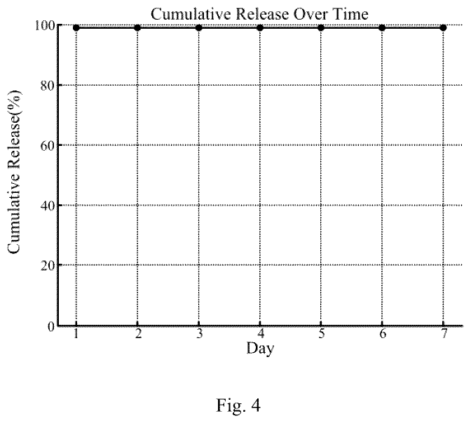

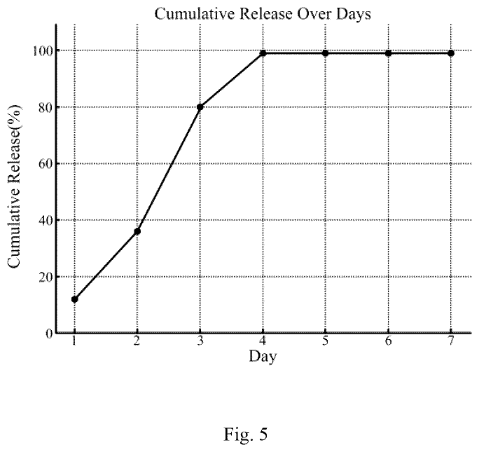

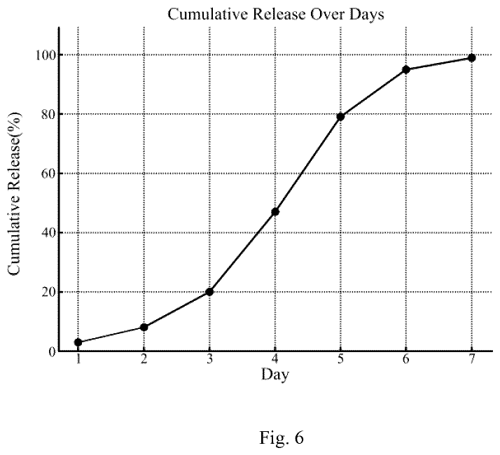

Please refer to FIGS. 4 to 6, which are schematic diagrams of the relationship between time and drug release degree when the nanofibers of the present invention are used as carriers without cross-linking, after cross-linking for 12 hours, and after cross-linking for 48 hours. As shown in FIG. 4, the nanofibers of the present invention cannot control the release of drugs when they are not cross-linked and used as carriers. As shown in FIG. 5, the nanofibers of the present invention are cross-linked for 12 hours. (Medium cross-linking) and then used as a carrier, which can slowly and linearly release the drug and maintain it for 4 days. Finally, as shown in FIG. 6, the nanofiber of the present invention is used as a carrier after 48 hours of cross-linking (high cross-linking). A carrier that releases the drug slowly and linearly for up to 7 days. It can be seen that when the nanofiber of the present invention is used as a carrier, the time and degree of drug release can be further controlled through different cross-linking times.

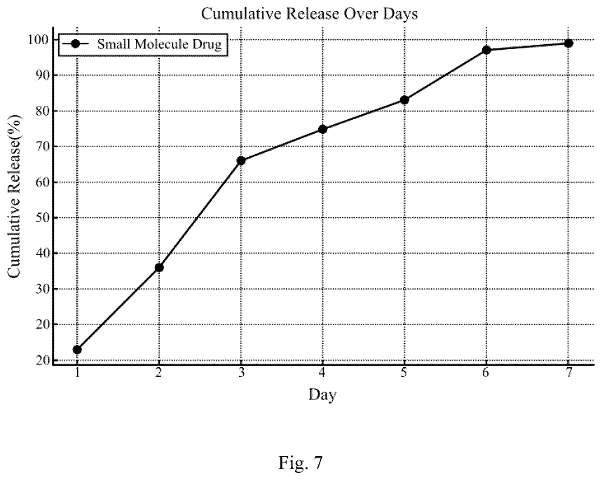

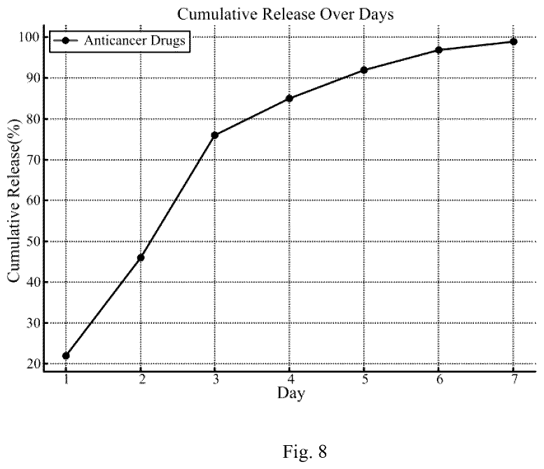

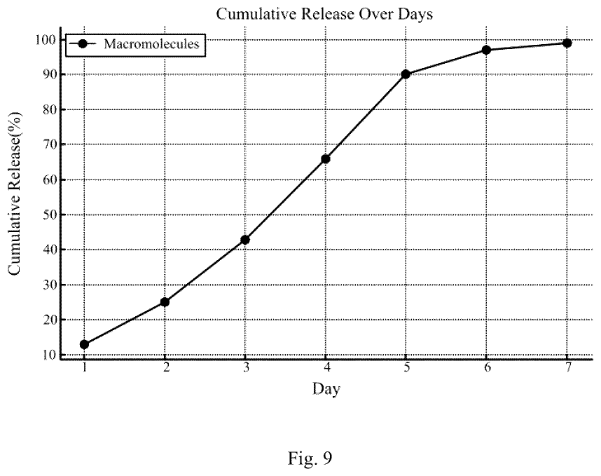

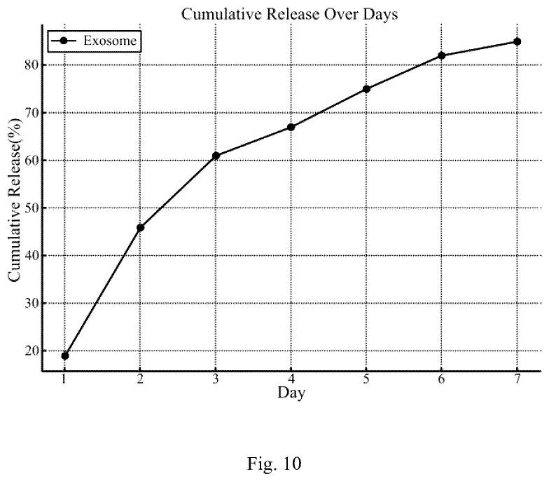

Please refer to FIGS. 7 to 10, which are schematic diagrams of the relationship between time and the release degree of small molecule drugs, macromolecule drugs and biological preparations using nanofibers cross-linked for 48 hours according to the present invention as carriers. As shown in FIG. 7, the nanofiber cross-linked for 48 hours of the present invention is used as a carrier, and the small molecule drug (MET) attached thereto can be slowly and linearly released within 7 days. As shown in FIG. 8, the nanofiber cross-linked for 48 hours of the present invention is used as a carrier to slowly and linearly release the small molecule drug (anti-cancer drug) attached to it within 7 days, which further includes two stages. The release rate is faster in the first 3 days, and then gradually slows down on the 3rd to 7th day. As shown in FIG. 9, the nanofiber cross-linked for 48 hours of the present invention is used as a carrier, and the macromolecule drug (growth factor) contained therein can be released slowly, evenly and linearly within 7 days. As shown in FIG. 10, the nanofiber cross-linked for 48 hours of the present invention is used as a carrier, and the biological agent contained therein can be released slowly, evenly and linearly within 7 days. It can be seen from this that when the nanofiber of the present invention is used as a carrier, the attached objects are diverse and various, and can accurately transport and control the release of drugs, biomolecules, genes, cells and nanoparticles, and are suitable for drug disease treatment, It has many uses such as tissue repair and regeneration engineering, wound treatment and cosmetic beauty.

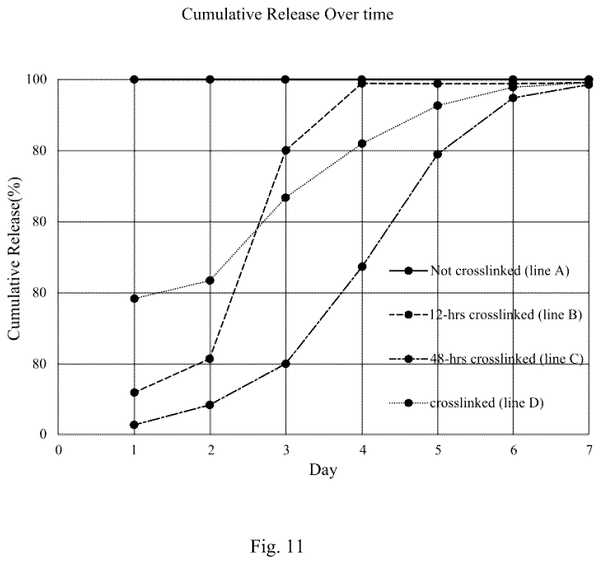

Please refer to FIG. 11, which is a schematic diagram of the relationship between cross-linking and bulk dynamic stability of the nanofibers of the present invention at different times. It can be seen from the figure that when non-cross-linked nanofibers are used as carriers, the drug is released immediately, as shown by line segment A. The 12-hour cross-linked nanofiber is used as a carrier to release the drug slowly and linearly for 4 days, as shown by line segment B. The 48-hour cross-linked nanofiber is used as a carrier to release the drug slowly and linearly for 7 days, as shown by line segment C. Finally, as shown by line segment D, it is a carrier composed of the uncrosslinked nanofibers, the 24-hour cross-linked nanofibers, and the 48-hour cross-linked nanofibers, which are mixed in a ratio of 1:1:1, it can be seen that the release degree is controlled between line segment B and line segment C. It can be seen from this that when the nanofiber of the present invention is used as a carrier, the time and degree of drug release can be further controlled through different cross-linking times or by mixing a variety of nanofibers with different cross-linking times in proportion to achieve in vivo dynamic stability of drug release.

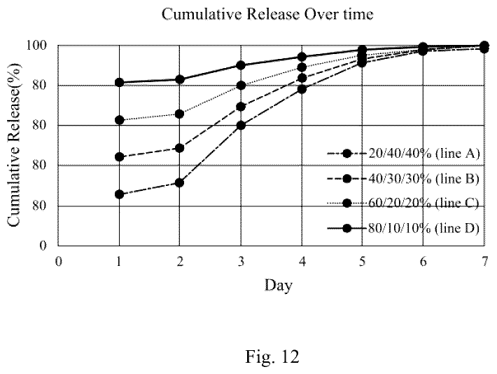

Please refer to FIG. 12, which is a schematic diagram of the relationship between the release degree of the nanofiber of the present invention loaded with the same bioactive material and the cross-linking combination ratio at different times. Notably, line segment A is a carrier composed of uncrosslinked nanofibers, 24-hour cross-linked nanofibers, and 48-hour cross-linked nanofibers, mixed in a ratio of 20:40:40. Line segment B is a carrier composed of uncrosslinked nanofibers, 24-hour cross-linked nanofibers, and 48-hour cross-linked nanofibers, mixed in a ratio of 40:30:30. Line segment C is a carrier composed of uncrosslinked nanofibers, 24-hour cross-linked nanofibers, and 48-hour cross-linked nanofibers, mixed in a ratio of 60:20:20. Line segment D is a carrier composed of uncrosslinked nanofibers, 24-hour cross-linked nanofibers, and 48-hour cross-linked nanofibers, mixed in a ratio of 80:10:10. As shown in the figure, when the proportion of uncrosslinked nanofibers is lower, the release rate of the bioactive materials is slower. Conversely, when the proportion of uncrosslinked nanofibers is higher, the release rate of the bioactive materials is faster. It can be seen from this that when the nanofibers of the present invention are used as carriers, the time and degree of the release of bioactive materials can be further controlled through different cross-linking times or by mixing a variety of nanofibers with different cross-linking times in proportion, so as to achieve in vivo dynamic stability of drug release of bioactive materials.

The above-mentioned embodiments only express some of the embodiments of the present invention, but they are not limitations of the patent scope of the present invention. For those with ordinary knowledge in this technical field, there are no limitations without departing from the patent scope of the present invention. Under the premise of maintaining the technical characteristics of the architecture, several deformations and improvements derived therefrom are within the scope of protection of the present invention.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A nanofiber carrier that contains biocompatible polymers and bioactive agents, and is electrospun to form the nanofiber carrier that has a three-dimensional structure, dynamic stability, and can be controlled along the time axis., wherein the nanofiber carrier is biologically active and provides rapid, stable, and controlled release with an uniform distribution.

2. The nanofiber carrier according to claim 1, further comprising an inorganic salt.

3. The nanofiber carrier according to claim 1, further comprising an adhesive forming agent, and the adhesive forming agent is a cross-linking agent.

4. The nanofiber carrier as described in claim 1, which has a release speed of constant speed, positioning, timing, rapid start and stable maintenance.

5. The nanofiber carrier as described in claim 1, the nanofiber carrier system is formed into a variety of different diameters through electrospinning to increase the reaction and carrying surface area.

6. The nanofiber carrier as described in claim 1, wherein the biocompatible polymer contains multi-source, multi-power nanostructures derived from a variety of different biopolymers.

7. The nanofiber carrier according to claim 1, wherein the biocompatible polymer is one or a combination of more than two of colloids, scaffolds, spheres, powders or films.

8. The nanofiber carrier as described in claim 1, wherein the biocompatible polymer is cross-linked by an enzyme, and the enzyme can be glutamine transaminase, lipase, lipase, One or a combination of more than two of peptidase, transpeptidase, oxidoreductase, tyrosinase, polyphenol oxidase, lacquer oxidase, peroxidase, and lysine oxidase.

9. The nanofiber carrier according to claim 1, wherein the biocompatible polymer is one of polysaccharide, protein or a combination of both.

10. The nanofiber carrier according to claim 1, wherein the average particle size of the biocompatible polymer is between 1 nm and 1 mm.

11. The nanofiber carrier according to claim 9, wherein the polysaccharide system is one or a combination of more than two of hyaluronic acid, alginic acid, and chitosan.

12. The nanofiber carrier according to claim 9, wherein the protein is one of gelatin, collagen, or a combination of more than two.

13. The nanofiber carrier according to claim 2, wherein the inorganic salt is hydroxyapatite, tricalcium phosphate, dicalcium phosphate, dicalcium phosphate dihydrate, tetracalcium phosphate, carbonic acid One or a combination of two or more salts, nitrates, sulfates, potassium salts, sodium salts and magnesium salts.

14. The nanofiber carrier according to claim 2, wherein the average particle size of the inorganic salt is between 1 nm and 1 mm.

15. The nanofiber carrier as described in claim 2, wherein the concentration of the biocompatible polymer is between 1 and 50% (w/v), and the concentration of the inorganic salt is between Between 1 and 50% (w/v), the bioactive agent is a hypoglycemic drug, the concentration is between 1 nM and 1M, and the concentration of the binder is between 1 and 50wt %.

16. The nanofiber carrier according to claim 1, wherein the bioactive agent is one or two of the transport and release effects of nanoparticles, drugs, growth factors, genes, biomolecules or cells. A combination of the above.

17. The nanofiber carrier as described in claim 1, wherein the bioactive agent is used in one or more of drug disease treatment, tissue repair, tissue engineering, wound treatment, cosmetics and beauty, or industry. combination of medicines.

18. The nanofiber carrier according to claim 17, wherein the drug disease treatment includes nerve regeneration, cancer treatment, diabetic wound treatment, muscle and tendon healing, cartilage degeneration treatment, bone healing or vertebral body shaping.

19. A method for preparing a nanofiber carrier according to claim 1, the steps include:
Mix 5~20 g of biocompatible polymer, 100 ml of sterile water for injection and 0.001~10 g of drug, heat to 40~80° C., and continue stirring for 30~240 minutes until the biocompatible polymer is dissolved. Form feed liquid;
Preheat the electrospinning device and maintain it at an ambient operating temperature of 35~60° C., a voltage of 20~100 kV, and a speed of the rotating receiver of the electrospinning device of 3~100 cm/min;
The peristaltic pump feeds the feed liquid into the electrospinning device at a speed of 1~30 ml/hour, and sprays it out of the collection paper on the rotating receiver through the spinning nozzle of the electrospinning device to form nanoparticles. rice fiber;
Separate the nanofibers from the collection paper;
The nanofiber, 1-ethyl-(3-dimethylaminopropyl)carbodiimide (EDC) and N-hydroxysuccinimide (NHS) were mixed into a cross-linked body, and the process was carried out for 12 to 72 seconds. hour cross-linking, with a ratio of nanofibers: 1-ethyl-(3-dimethylaminopropyl)carbodiimide (EDC): N-hydroxysuccinimide (NHS) is 1:
0.5~5:0.05~0.52, and prepare it with absolute alcohol and ensure that the nanofibers are completely submerged in the cross-linking solution. Clean and remove the cross-linking solution with absolute alcohol, and repeat 3 times, each time for 1 to 4 hours;
After washing once with pure water, pre-freeze at −80° C. for 24 to 72 hours; and
Freeze-dry for 24 to 72 hours to form a nanofiber carrier.

20. A nanofiber carrier according to claim 1, which can be used in drug disease treatment, tissue repair, tissue engineering, wound treatment, cosmetics and beauty, or industry.
